(19) United States
(12) Patent Application Publication
(10) Pub. No.: US 2019/0321560 A1
(43) Pub. Date: Oct. 24, 2019

(54) HYPODERMIC INJECTION ASSEMBLY

(71) Applicant: Lisa Lawder, Stoney Creek (CA)

(72) Inventor: Lisa Lawder, Stoney Creek (CA)

(21) Appl. No.: 15/956,879

(22) Filed: Apr. 19, 2018

(51) Int. Cl. A61M 5/315; A61M 5/34; A61M 5/31

(52) U.S. Cl. CPC A61M 5/31578; A61M 5/34; A61D 7/00; A61M 2250/00; A61M 5/3148

(57) ABSTRACT

A hypodermic injection assembly for treating a canine for an allergic reaction includes a reservoir that contains a fluid allergy medication for a canine. A hypodermic needle is fluidly coupled to the reservoir for piercing skin on a canine. A plunger is slidably positioned in the reservoir. The plunger is urged toward the hypodermic needle to urge the fluid allergy medication outwardly through the hypodermic needle. In this way the hypodermic needle injects the fluid allergy medication into a canine when the canine is experiencing an allergic reaction. A container is provided and each of the reservoir, the hypodermic needle and the plunger are positioned in the container for transportation.

### CROSS-REFERENCE TO RELATED APPLICATIONS

Not Applicable

### STATEMENT REGARDING FEDERALLY SPONSORED RESEARCH OR DEVELOPMENT

Not Applicable

### THE NAMES OF THE PARTIES TO A JOINT RESEARCH AGREEMENT

Not Applicable

### INCORPORATION-BY-REFERENCE OF MATERIAL SUBMITTED ON A COMPACT DISC OR AS A TEXT FILE VIA THE OFFICE ELECTRONIC FILING SYSTEM

Not Applicable

### STATEMENT REGARDING PRIOR DISCLOSURES BY THE INVENTOR OR JOINT INVENTOR

Not Applicable

### BACKGROUND OF THE INVENTION

### (1) Field of the Invention

### (2) Description of Related Art Including Information Disclosed Under 37 CFR 1.97 and 1.98

The disclosure and prior art relates to injection devices and more particularly pertains to a new injection device for treating a canine for an allergic reaction.

### BRIEF SUMMARY OF THE INVENTION

An embodiment of the disclosure meets the needs presented above by generally comprising a reservoir that contains a fluid allergy medication for a canine. A hypodermic needle is fluidly coupled to the reservoir for piercing skin on a canine. A plunger is slidably positioned in the reservoir. The plunger is urged toward the hypodermic needle to urge the fluid allergy medication outwardly through the hypodermic needle. In this way the hypodermic needle injects the fluid allergy medication into a canine when the canine is experiencing an allergic reaction. A container is provided and each of the reservoir, the hypodermic needle and the plunger are positioned in the container for transportation.

There has thus been outlined, rather broadly, the more important features of the disclosure in order that the detailed description thereof that follows may be better understood, and in order that the present contribution to the art may be better appreciated. There are additional features of the disclosure that will be described hereinafter and which will form the subject matter of the claims appended hereto.

The objects of the disclosure, along with the various features of novelty which characterize the disclosure, are pointed out with particularity in the claims annexed to and forming a part of this disclosure.

### BRIEF DESCRIPTION OF SEVERAL VIEWS OF THE DRAWING(S)

The disclosure will be better understood and objects other than those set forth above will become apparent when consideration is given to the following detailed description thereof. Such description makes reference to the annexed drawings wherein:

FIG. 1 is a perspective view of a reservoir, hypodermic needle and a plunger of a hypodermic injection assembly according to an embodiment of the disclosure.

FIG. 2 is a perspective view of an embodiment of the disclosure.

FIG. 3 is a cross sectional view taken along line 3-3 of FIG. 1 of an embodiment of the disclosure.

FIG. 4 is a perspective view of a plurality of reservoirs, hypodermic needles and plungers of an embodiment of the disclosure.

### DETAILED DESCRIPTION OF THE INVENTION

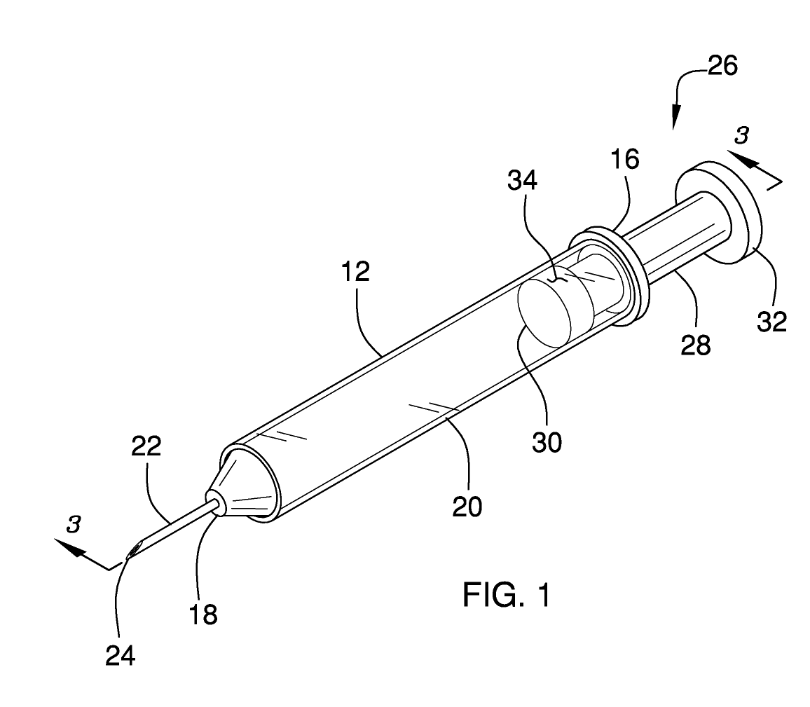

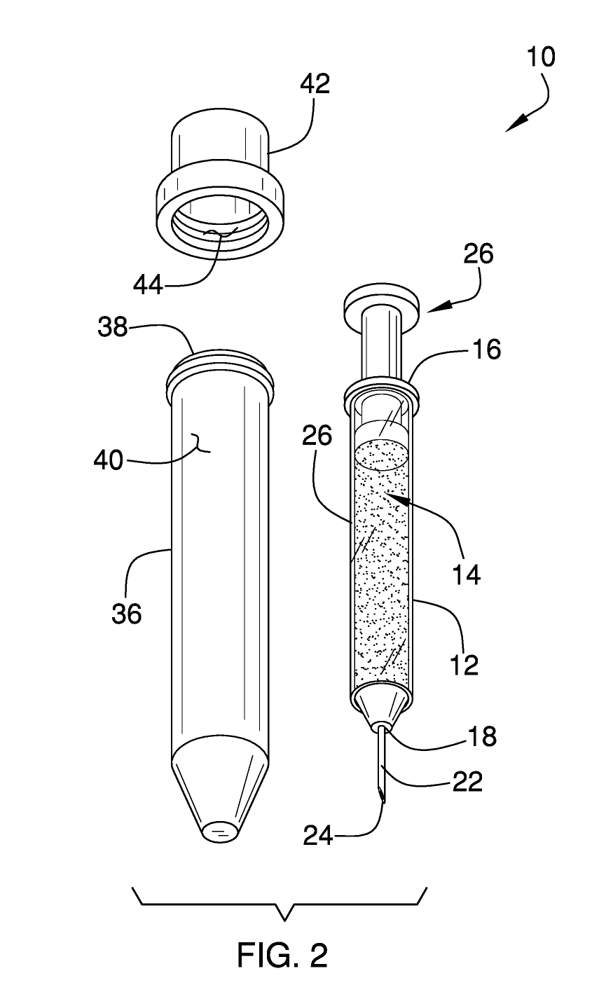

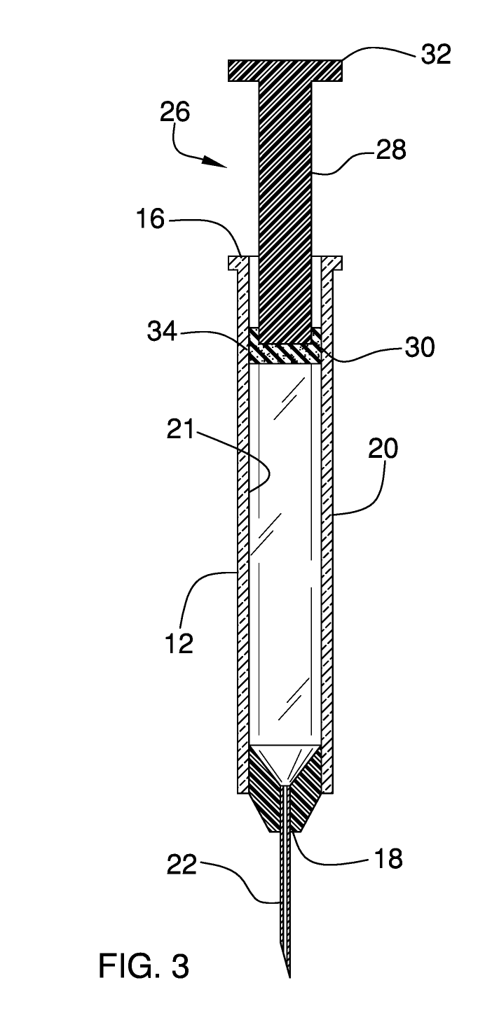

With reference now to the drawings, and in particular to FIGS. 1 through 4 thereof, a new injection device embodying the principles and concepts of an embodiment of the disclosure and generally designated by the reference numeral 10 will be described.

As best illustrated in FIGS. 1 through 4, the hypodermic injection assembly 10 generally comprises a reservoir 12 that contains a fluid allergy medication 14 for a canine. The reservoir 12 has a first end 16, a second end 18 and an outer wall 20 extending therebetween. The outer wall 20 has an inside surface 21 and the outer wall 20 is continuous such that the reservoir 12 has a cylindrical shape. Each of the first end 16 and the second end 18 is open and the outer wall 20 tapers inwardly toward the second end 18. Thus, the second end 18 has a diameter that is less than a diameter of the first end 16. Additionally, the reservoir 12 is comprised of a translucent material such that a level of the fluid allergy medication 14 is visible through the outer wall 20. The fluid allergy medication 14 may be epinephrine or other sympathomimetic catecholamine medication.

A hypodermic needle 22 is fluidly coupled to the reservoir 12 for piercing skin on a canine. The hypodermic needle 22 is positioned in the second end 18 of the reservoir 12 and the hypodermic needle 22 has a distal end 24 with respect to the second end 18 of the reservoir 12. The distal end 24 is open such that the fluid allergy medication 14 may pass through the distal end 24. The hypodermic needle 22 may have a length and a gauge that is common to canine hypodermic needles.

A plunger 26 is provided and the plunger 26 is slidably positioned in the reservoir 12. The plunger 26 is urged toward the hypodermic needle 22 thereby facilitating the plunger 26 to urge the fluid allergy medication 14 outwardly through the hypodermic needle 22. In this way the hypodermic needle 22 injects the fluid allergy medication 14 into a canine when the canine is experiencing an allergic reaction. The canine may be a domesticated dog of any size and breed that is a family pet or the like.

The plunger 26 includes a stem 28 extending between a disk 30 and a cap 32. The stem 28 extends through the first end 16 of the reservoir 12 having the disk 30 being positioned within the reservoir 12 and having the cap 32 being positioned outside of the reservoir 12. The disk 30 has an outer surface 34 and the outer surface 34 frictionally engages the inside surface 21 of the outer wall 20 of the reservoir 12. Moreover, the disk 30 is comprised of a resiliently compressible material, such as rubber or the like, such that the outer surface 34 of the disk 30 forms a fluid impermeable seal with the inside surface 21 of the outer wall 20 of the reservoir 12.

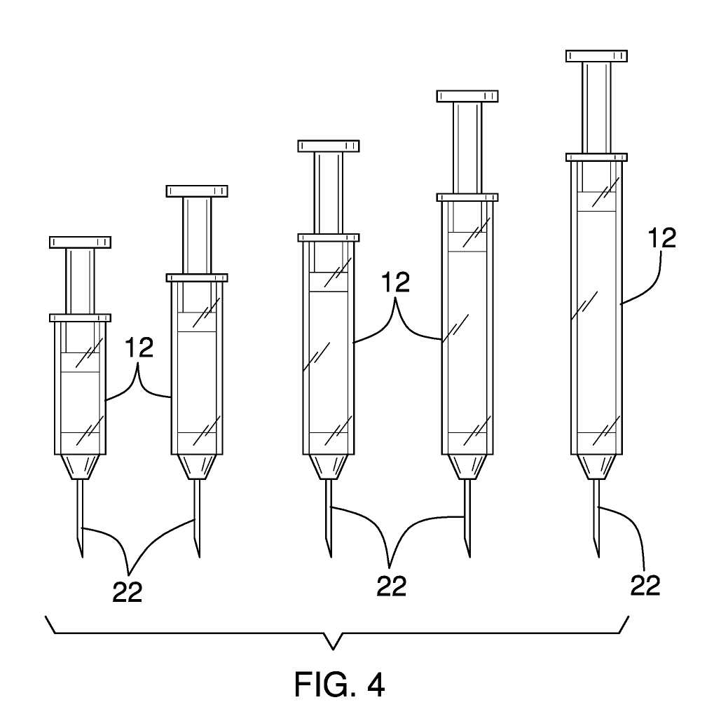

The cap 32 abuts the first end 16 of the reservoir 12 when the plunger 26 is urged into the reservoir 12 and the stem 28 has a length that corresponds to a predetermined volume of fluid that the plunger 26 can urge. Thus, the plunger 26 urges the predetermined volume of the fluid allergy medication 14 outwardly through the hypodermic needle 22, and the predetermined volume corresponds to a recommended dosage for the canine based on size, weight and breed. The reservoir 12 may have a fluid capacity ranging between approximately 1.0 ml and 15.0 ml thereby facilitating the reservoir 12 to accommodate the recommended dosage for the canine. As shown in FIG. 4, a plurality of reservoirs 12, hypodermic needles 22 and plungers 26 may be provided. Each of the reservoirs 12 as shown in FIG. 4 may have a unique fluid volume with respect to each other. Thus, a variety of breeds, sizes and weights of canines can be treated for allergic reactions.

A container 36 is provided and each of the reservoir 12, the hypodermic needle 22 and the plunger 26 are positioned in the container 36 for transportation. The container 36 is comprised of a rigid material to inhibit the hypodermic needle 22 from piercing the container 36. In this way the container 36 inhibits the hypodermic needle 22 from inadvertently piercing a user or the canine. The container 36 has a primary end 38 and an outside surface 40. The primary end 38 is open to receive the reservoir 12, the hypodermic needle 22 and the plunger 26, and the outside surface 40 is threaded adjacent to the primary end 38. A lid 42 is provided to threadably engage the container 36 and retain each of the reservoir 12, the hypodermic needle 22 and the plunger 26 within the container 36. The lid 42 has an interior surface 44 and the interior surface 44 threadably engages the outside surface 40 of the container 36 to releasably retain the lid 42 on the container 36.

In use, the container 36 is a carried by a pet owner when the pet owner is travelling away from home or otherwise in a remote location with respect to known veterinary clinics. The reservoir 12 is removed from the container 36 when the canine experiences an allergic reaction to a bee sting, contact with a plant and any other type of allergic reaction. The hypodermic needle 22 is extended into the skin of the canine and the plunger 26 is depressed unit the cap 32 on the plunger 26 abuts the second end 18 of the reservoir 12. Thus, the predetermined amount of the fluid allergy medication 14, determined by a veterinarian or the like, is injected into the canine to treat the allergic reaction.

With respect to the above description then, it is to be realized that the optimum dimensional relationships for the parts of an embodiment enabled by the disclosure, to include variations in size, materials, shape, form, function and manner of operation, assembly and use, are deemed readily apparent and obvious to one skilled in the art, and all equivalent relationships to those illustrated in the drawings and described in the specification are intended to be encompassed by an embodiment of the disclosure.

Therefore, the foregoing is considered as illustrative only of the principles of the disclosure. Further, since numerous modifications and changes will readily occur to those skilled in the art, it is not desired to limit the disclosure to the exact construction and operation shown and described, and accordingly, all suitable modifications and equivalents may be resorted to, falling within the scope of the disclosure. In this patent document, the word “comprising” is used in its non-limiting sense to mean that items following the word are included, but items not specifically mentioned are not excluded. A reference to an element by the indefinite article “a” does not exclude the possibility that more than one of the element is present, unless the context clearly requires that there be only one of the elements.

I claim:

1. A hypodermic injection assembly containing a fluid allergy medication for a canine wherein said assembly is configured to inject the fluid allergy medication into a canine to stop an allergic reaction in the canine, said assembly comprising:
a reservoir containing a fluid allergy medication for a canine;
a hypodermic needle being fluidly coupled to said reservoir wherein said hypodermic needle is configured to pierce skin on a canine;
a plunger being slidably positioned in said reservoir, said plunger being urged toward said hypodermic needle such that said plunger urges said fluid allergy medication outwardly through said hypodermic needle wherein said hypodermic needle is configured to inject said fluid allergy medication into a canine when the canine is experiencing an allergic reaction; and
a container having said reservoir, said hypodermic needle and said plunger positioned therein for transporting said reservoir, said hypodermic needle and said plunger.

2. The assembly according to claim 1, wherein said reservoir has a first end, a second end and an outer wall extending therebetween, said outer wall having an inside surface, said outer wall being continuous such that said reservoir has a cylindrical shape, each of said first end and said second end being open, said outer wall tapering inwardly toward said second end such that said second end has a diameter being less than a diameter of said first end, said reservoir being comprised of a translucent material such that a level of said fluid allergy medication is visible through said outer wall.

3. The assembly according to claim 2, wherein said hypodermic needle is positioned in said second end of said reservoir, said hypodermic needle having a distal end with respect to said second end of said reservoir, said distal end being open such that said fluid allergy medication may pass through said distal end.

4. The assembly according to claim 2, wherein said plunger includes a stem extending between a disk and a cap, said stem extending through said first end of said reservoir having said disk being positioned within said reservoir and having said cap being positioned outside of said reservoir, said disk having an outer surface, said outer surface frictionally engaging said inside surface of said outer wall of said reservoir such that said outer surface of said disk forms a fluid impermeable seal with said inside surface of said outer wall of said reservoir.

5. The assembly according to claim 4, wherein said cap abuts said first end of said reservoir wherein said plunger is urged into said reservoir such that said plunger urges a predetermined amount of said fluid allergy medication outwardly through said hypodermic needle that corresponds to a recommended dosage for the canine based on size, weight and breed.

6. The assembly according to claim 1, wherein said container has a primary end and an outside surface, said primary end being open to receive said reservoir, said hypodermic needle and said plunger, said outside surface being threaded adjacent to said primary end.

7. The assembly according to claim 6, further comprising a lid threadably engaging said container to retain each of said reservoir, said hypodermic needle and said plunger within said container, said lid having an interior surface, said interior surface threadably engaging said outside surface of said container to releasably retain said lid on said container.

8. A hypodermic injection assembly containing a fluid allergy medication for a canine wherein said assembly is configured to inject the fluid allergy medication into a canine to stop an allergic reaction in the canine, said assembly comprising:
a reservoir containing a fluid allergy medication for a canine, said reservoir having a first end, a second end and an outer wall extending therebetween, said outer wall having an inside surface, said outer wall being continuous such that said reservoir has a cylindrical shape, each of said first end and said second end being open, said outer wall tapering inwardly toward said second end such that said second end has a diameter being less than a diameter of said first end, said reservoir being comprised of a translucent material such that a level of said fluid allergy medication is visible through said outer wall;
a hypodermic needle being fluidly coupled to said reservoir wherein said hypodermic needle is configured to pierce skin on a canine, said hypodermic needle being positioned in said second end of said reservoir, said hypodermic needle having a distal end with respect to said second end of said reservoir, said distal end being open such that said fluid allergy medication may pass through said distal end;
a plunger being slidably positioned in said reservoir, said plunger being urged toward said hypodermic needle such that said plunger urges said fluid allergy medication outwardly through said hypodermic needle wherein said hypodermic needle is configured to inject said fluid allergy medication into a canine when the canine is experiencing an allergic reaction, said plunger including a stem extending between a disk and a cap, said stem extending through said first end of said reservoir having said disk being positioned within said reservoir and having said cap being positioned outside of said reservoir, said disk having an outer surface, said outer surface frictionally engaging said inside surface of said outer wall of said reservoir such that said outer surface of said disk forms a fluid impermeable seal with said inside surface of said outer wall of said reservoir, said cap abutting said first end of said reservoir wherein said plunger is urged into said reservoir such that said plunger urges a predetermined amount of said fluid allergy medication outwardly through said hypodermic needle that corresponds to a recommended dosage for the canine based on size, weight and breed;
a container having said reservoir, said hypodermic needle and said plunger positioned therein for transporting said reservoir, said hypodermic needle and said plunger, said container being comprised of a rigid material to inhibit said hypodermic needle from piercing said container wherein said container is configured to inhibit said hypodermic needle from inadvertently piercing a user, said container having a primary end and an outside surface, said primary end being open to receive said reservoir, said hypodermic needle and said plunger, said outside surface being threaded adjacent to said primary end; and
a lid threadably engaging said container to retain each of said reservoir, said hypodermic needle and said plunger within said container, said lid having an interior surface, said interior surface threadably engaging said outside surface of said container to releasably retain said lid on said container.
